(19) United States
(12) Patent Application Publication
(10) Pub. No.: US 2010/0270391 A1
(43) Pub. Date: Oct. 28, 2010

(54) OIL LAMP WITH HEAT CONDUCTIVE ELEMENT

(76) Inventors: Gerald Allison, East Windsor, NJ (US); Lyse Tranzeat, West Drayton (GB); Jonathan Williams, London (GB)

Correspondence Address: WINSTON & STRAWN LLP;PATENT DEPARTMENT, 1700 K STREET, N.W., WASHINGTON, DC, 20006, US

(21) Appl. No.: 12/728,880

(22) Filed: Mar. 22, 2010

Related U.S. Application Data
(60) Provisional application No. 61/173,369, filed on Apr. 28, 2009.

(51) Int. Cl. A61L 9/03

(52) U.S. Cl. 239/44

(57) ABSTRACT

The present invention relates to a device generally in the form of an oil lamp and adapted to a dual function of diffusing light and a volatile material. The device includes a fuel and a wick formed of combustible material, and a heat conductive element adapted to receive a gel or solid material capable of melting at the burning temperatures of the device. The material is a carrier for a volatile substance that is released into the atmosphere upon melting of the carrier material. Fragrances are preferred embodiments of the volatile substance. Methods for use of the device in the simultaneous diffusion of light and volatile substances, namely fragrances, are also disclosed.

This application claims the benefit of application No. 61/173,369 filed Apr. 28, 2009.

### TECHNICAL FIELD

The present invention relates generally to oil lamps of the type that comprise a fluid vessel containing a liquid, gel or solid fuel and a wick formed of combustible material. It concerns more particularly an oil lamp of this type which further comprises a heat conductive element adapted to receive a gel or solid material capable of melting at the burning temperatures of the lamp. Said material is a carrier for a volatile substance that is released into the atmosphere upon melting of the carrier material. Fragrances are preferred embodiments of the volatile substance. Methods for use of the invention's oil lamp in the simultaneous diffusion of light and volatile substances, namely fragrances, are also disclosed.

### BACKGROUND

The diffusion of volatile substances via vaporization of the latter from a carrier material that is submitted to a heat source is well known from the prior art. Some of the most common forms of such devices are scented candles and oil lamps that function through the heating of liquid scented oils. Typical such known devices are also generally discussed for example in U.S. Pat. No. 5,840,246, to Randall L. Hammons (see for example column 1 and top of column 2). This prior art reference further describes such a device which is further equipped with a porous surface that both absorbs and diffuses a fragranced oil and is warmed up through the head dispersed by the lamp's combustible wick. The porous surface is solid but does not melt and cannot therefore be entirely consumed.

In U.S. Pat. Nos. 6,802,707, 6,780,382 and 7,247,018, all assigned to S.C. Johnson & Son, Inc., there are described candles provided with heat conductive elements intended to distribute heat from a burning flame at a wick to a simmer or melting plate, so as to increase the speed at which a solid meltable fragranced fuel, such as a candle material, supported by the plate or in close contact therewith, becomes liquid and releases the fragrance.

These prior art devices cannot however accommodate the use of both a meltable fuel and a liquid fuel, arranged as described hereinafter.

U.S. Pat. No. 6,333,009 describes an oil lamp adapted to disperse a fragrance contained in the heating oil which also feeds the burning wick. The oil container is provided with vents which allow evaporation of the fragrance as the oil is heated through a heat transfer system. This device avoids the severe heating of the fragrance which is typical of burning lamps of a conventional type, but it does not allow replacement of the fragrance separately from the oil supply to the wick. Moreover, as the fragrance is containing in the wick feeding oil, part of the fragrance is still burned at the wick.

The present invention aims at providing a novel combination of a device capable of diffusing into its surroundings a fragrance, or other volatile active ingredient, and also having a light diffusing capability, but wherein the fragrance can be replaced separately from the fuel oil and can be entirely consumed independently of the latter. The device according to the invention provides more effective control of the burning process and of the fragrance diffusion into the surroundings.

### SUMMARY OF THE INVENTION

The present invention now relates to a device for the diffusion of light and of an active volatile substance, comprising a reservoir adapted to carry a liquid or solid fuel intended as an oil supply for a combustible wick, said reservoir being provided with a capping piece having a hole or aperture adapted to receive a lodging element for the combustible wick, said wick being partially contained in the solid or liquid fuel and having an upper end intended for providing the flame and extending from the cap's hole and, wherein:
i) the device further comprises a melting plate for carrying a meltable material containing said active volatile substance, said plate being lodged on top of the reservoir's capping piece;
ii) the device comprises a reflux protection element lodged across the hole or aperture of the capping piece and shaped so as to entirely cover the capping piece's hole and prevent liquid reflux into the reservoir; and
iii) a conductive element is provided in the near vicinity of the upper end of the wick which provides the flame and maintained in direct contact with the melting plate so as to provide conduction heat thereto.

According to preferred embodiments of the invention, the active volatile substance is carried only in the meltable material or substance and is not contained in the fuel that serves to light up the device. The meltable substance or material can be a solid or a gel candle material and the active volatile substance is preferably a fragrance.

The melting plate is preferably of a conical, concave or slanting shape so as to form a combustion chamber with enough depth to centralize the melted fluid around the device's zone of heat provided by the burning wick flame.

The device comprises a fuel element, typically a liquid oil, and a container for the fuel oil. The container comprises a melting plate or heat conductive element which is in direct contact with a zone of the device where heat is generated by a flame generated by a burning wick. The latter is fed in the fuel contained in the reservoir and is supported by a structure comprising a conductive element in direct contact with the melting plate.

The melting plate acts both as a chamber for a meltable material containing the active volatile substance that one desires to diffuse into the surroundings of the device, and as a heat conducting element allowing the meltable material to quickly melt as the wick burns, so as to provide for evaporation of the volatile substance from the melted material. The melting plate is therefore shaped so as to concentrate the melted materials into its lower part which is in direct contact with the zone heated by the burning wick. Therefore, the melting plate has preferably a concave or conical shape of enough depth to allow the fuel to entirely melt within the chamber formed by the melting plate.

By an “active volatile substance or material”, or a “volatile substance or material”, all of which are used here interchangeably, we mean here a substance that is at least partially volatile, i.e. can evaporate under atmospheric pressure and normal room temperatures typically comprised between 15 and 35° C., and which is able to impart a benefit to the surroundings of the device according to the invention, and in particular to improve the quality of the air in the room or space where the device is located. According to advantageous embodiments of the invention, the active volatile is selected amongst the group of fragrance, deodorizing or malodour counteracting, sanitising, insect repellent compositions, and their mixtures. It is clear however that other volatile or partially volatile substances may be diffused into the atmosphere from the device of the invention.

Preferably the active volatile substance is a fragrance or a malodour counteracting composition.

The active volatile substance is a composition which contains at least one active ingredient. Said ingredient is capable of imparting a benefit to the surrounding space or enclosed space in which the device is activated, and may be accompanied by optional ingredients which can be beneficial to said active volatile material. In other words the active composition contains an active volatile material, comprising at least one ingredient, and optionally one or more ingredients selected from the group consisting of solvents, thickeners, anti-oxidants, dyes, bittering agents and UV inhibitors.

As the active volatile material, there can be used preferably a perfume. Other suitable active volatile materials can be deodorizing or sanitizing agents or insect repellents or any other active materials capable of imparting perceptible and desirable benefits to the quality of the air into which they are diffused.

As perfume there can be used any ingredient or mixture of ingredients currently used in perfumery, i.e. capable of exercising a perfuming action, meaning modifying or imparting the odour of the surrounding air. This means that a malodour counteracting composition, capable of reducing or suppressing a large variety of malodours, such as body malodour, tobacco malodour, kitchen or bathroom malodour for example, are also understood herein as being comprised in the “perfume”, “fragrance” or “perfuming composition” definition. Often, such a perfuming composition will be a more or less complex mixture of ingredients of natural or synthetic origin. The nature and type of said ingredients do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpene hydrocarbons, nitrogenous or sulphurous heterocyclic compounds and essential oils of natural or synthetic origin. Many of these ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. Many are known to possess malodour counteracting and/or antibacterial activity so that, in addition to being capable of perfuming, and thus imparting a pleasant smell to, the surrounding air, they also help purify and sanitize the latter, and/or remove any malodour (i.e. unpleasant smell) thereof.

Natural oils such as lavender, cedar, lemon and other essential oils and extracts are particularly preferred for advantageous embodiments of the invention.

Although special mention has been made hereinabove of the perfuming effect that can be exerted by the devices of the invention, the same principles apply to analogous devices for the diffusion of deodorizing or sanitizing vapors, the perfume being replaced by a deodorizing composition, an antibacterial, an insecticide, an insect repellent or an insect attractant, or a so-called mothproofer device. By the term “sanitizing vapors”, we refer here not only to the vapors of those substances which can enhance the degree of acceptance of the air surrounding the observer, but also to those substances which can exert an attractant or repellent effect towards certain species of insects, for instance towards houseflies or mosquitoes, or else, which can have bactericide or bacteriostatic activity. It goes without saying that mixtures of such agents can also be used.

The total amount of active volatile material in the meltable substance may be comprised between 5% and 100%, preferably between 30% and 70%, of the weight of the meltable material contained in the melting plate.

The meltable material shall preferably be a candle type material, preferably a gel or wax. The wax can be formed of paraffin, soy wax, soy/paraffin mixture, montan wax, carnauba wax, beeswax, microcrystalline wax, fatty acid and fatty acid ester based such as the materials described in International publication WO 2005/079219 for example, in particular di-trimethylol propane tetrastearate. Of course mixtures of such waxes can also form the meltable substance provided that they are compatible with the use in combination with the active volatile substance, particularly fragrances.

As the meltable material, gels can also be used. Polyamide gels, namely those provided by Arizona Chemical under the tradename of SLYVACLEAR®, are advantageous embodiments thereof.

Other materials that can be used as the meltable substance are well-known to the skilled person and described in prior documents such as for example U.S. Pat. No. 6,802,707 previously cited. The meltable substance may take any desired shape such as pucks, donuts, balls, pellets, slivers, particulates, wafers, disks, or any other shape that is compatible with the melting plate shape. Particularly attractive shapes are particulate type forms, such as balls and pellets, which make it possible to easily replenish the melted substance upon total consumption of the previous supply. This also facilitates varying the nature of the fragrance over time. Of course, it is also possible to use colored particles which render the device particularly attractive from an esthetic point of view.

The melting plate is formed of any material having good thermal conductivity properties, for example metals, ceramic and glass materials, or yet combinations of these. Aluminum, copper iron, tin, brass, platinum, silver and gold, or even glass, and more preferably aluminum, are quite convenient materials therefor. As previously mentioned, the melting plate is concave and has preferably a depth typically comprised between 2 and 5 cm. The diameter shall typically be from 5 to 15 cm. It is however clear that these values are not limiting and that the skilled person is quite able to modify them so as to adapt the device to the desired performance, as a function of the amount and shape of the meltable material that one desires to use.

The melting plate is typically in direct contact with the heat conductive elements capable of transmitting the heat generated by the flame to the melting plate. Typically such elements will be formed by a structure which sustains the wick and helps centralize the wick and the flame during combustion. The structure will be formed typically of a cylindrically shaped metal tube which is hollow and traversed by the wick and preferably formed of aluminum, combined with a supporting metallic element lodged in the upper aperture of the container carrying the fuel oil supply for the wick.

Suitable combustion oils are well-known to the skilled person and formed of the materials usually employed in conventional oil lamps. Specific examples include fuels from either synthetic or natural origin (bio-fuels). The synthetic fuels will include ISOPAR® G, L, H, M and V. Most liquid petroleum distillates are also convenient for this purpose. ISOPAR® M is a preferred synthetic fuel.

Regarding bio-fuels, fatty acid esters, fatty acids and natural plant oils will be preferred choices. Fatty acid esters will include methyl laurate, methyl soyate, methyl plamitate and ethyl laurate. Preferred fatty acids will include oleic acid and linoleic acid.

Most natural plant base oils, such as almond oil, sunflower oil and apricot seed oil, are all appropriate sources of combustion fuel.

The most preferred naturally derived blended fuels of fatty acid and fatty acid ester for the lamp according to the invention are mixtures of ethyl laurate and oleic acid, preferably in a relative ratio of 2:1 respectively.

Conventional wick materials, appropriate for the purposes of the invention, include cotton, paper and nylon based materials, with a preference for cotton made wicks.

The combustion fuel that feeds the wick and makes it possible to generate the flame may be perfumed, but preferred embodiments of the invention will only contain fragrance in the meltable material contained in the melting place. This advantageously avoids having “burnt” fragrance notes diffused into the devices surroundings.

The combustion fuel is contained in a reservoir or container which may be of any appropriate shape and material. A more detailed description of such containers is not warranted here as they are well-known to the person skilled in the art of oil lamps. Moreover, it is clear that containers such as the one described in previously mentioned U.S. Pat. No. 6,333,009 can be combined with a melting plate as here-described to provide alternative embodiments of the device possibly diffusing two types of fragrances simultaneously if desired.

According to the invention, the melting plate shall be lodged on the upper aperture of the fuel container in a manner allowing to be heated by conduction by the flame of the oil lamp. As explained in more detail with reference to the figures below, the melting plate shall preferably be lodged on top of the fuel container, preferably lodged on its capping part, and be crossed through by the wick and its supporting structure, including the heat conducting elements. In this manner, the fragranced melt obtained from the meltable material shall be collected at the bottom of the melting plate and centered in the zone in close contact with the heating flame, so as to ensure constant and uniform diffusion of the fragrance.

In order to avoid reflux of the melted fragrance-containing substance into the combustion fuel container, the device according to the invention also comprises a further essential consisting of a reflux protection element shaped so as to entirely cover the capping piece's hole or aperture and prevent liquid reflux into the reservoir. Such reflux protection element shall be typically made of a heat conductive material so as to further improve the transfer of heat to the melting material and can form the upper part of the wick lodging structure. It goes without saying that the capping piece of the fuel container, the melting plate, the cylindrical lodging element for the wick and the reflux protection element may all be separate pieces or be part of a single piece, which may optionally be sold separately from the burning fuel or gel, from the scented wax, as a refillable piece.

The device according to the invention may be commercialized in an assembled form or in the form of a kit having its various elements separate and adapted to be assembled by the user.

It may be provided with a unique design and has a dual functionality, acting as a light source (lamp oil only) or as a light source and as a simmering/candle wax burner, the melting plate serving as the reservoir for scented materials in particular, which can be shaped in colored in attractive forms so as to furnish a decorative device.

### BRIEF DESCRIPTION OF THE DRAWINGS

Preferred embodiments of the invention are illustrated by the appended drawing figures, wherein:

FIG. 1 represents a cross-section view of an embodiment of the device showing all the parts assembled;

FIG. 2 represents in cross-section and partially exploded view, in more detail, the wick and supporting structure therefor, as well as the heat conduction pieces; and

FIG. 3 represents an elevated view of an operating device according to the invention.

### DETAILED DESCRIPTION OF THE PREFERRED EMBODIMENTS

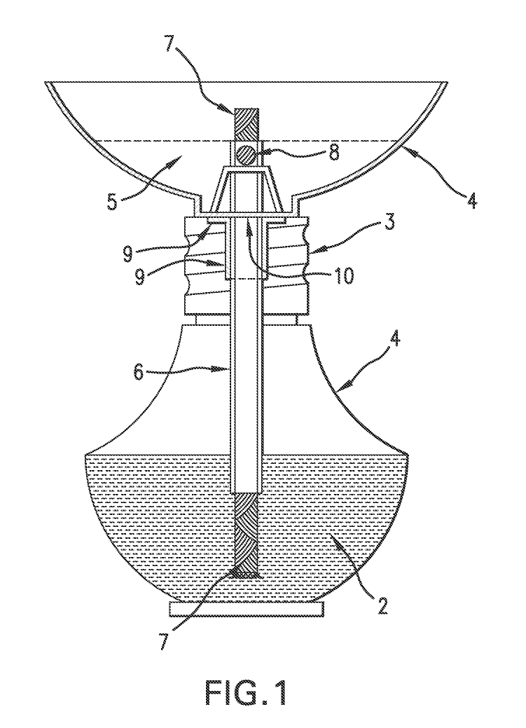

With reference to FIG. 1, representing an embodiment of the invention, the device comprises a container 1 for a combustion fuel 2, provided with an upper aperture capped by a piece 3. A concave shaped melting plate 4 is lodged on top of the capping piece 3 and contains a melted material 5 carrying a fragrance or other volatile material. A hollow cylindrical tube 6 crosses through an aperture or hole provided in the capping piece 3 so as to extend between the fuel 2 and the upper end of the wick 7 and serves as the supporting or guiding structure for the latter during the combustion. The upper end of the wick 7 provides the lighting flame which also serves as the heat source for maintaining the meltable substance 5 in a continuously melted state and thus diffuses the volatile substance contained in said meltable material into the surroundings.

The wick holding tube 6 which extends from the bottom of the lamp to the top of the melting plate 4 keeps the wick centralized and its top section will also help to keep the flame at the center of the device's structure. This top section is provided with four ventilation holes 8, approximately 0.1-4 mm in diameter and approximately 0.2-1 mm distance away from each other, the function of which is to ensure proper oxygen flow and combustion and a steady supply of thermal energy to the melting plate. The overall continuous capillary fuel driving process up the wick also depends on these holes in order to main proper fuel supply to the wick and a consistent flame height throughout the burning process.

The wick holding tube is typically from 1.5 to 25 cm long, depending on the size of the lamp and more preferably its length will be comprised between 5 and 10 cm, but both the diameter and the length of the tube will vary as a function of the shape and size of the device.

An inverted or washer like valve 9 is inserted inside of the lamp oil container capping piece 3 and its main function is to prevent reflux of the melted gel or wax 5 from the melting plate into the lamp fuel 2. This washer like valve is an over fitting glove—like tube, that is tightly bound to the main wick holding tube 6 and is shaped in a manner ensuring a complete seal of the capping piece aperture or hole 10 through its upper part 9′.

This washer like valve will extend from the top of the lamp oil cap, about 1-4 centimeter downward, interlocking over the primary wick holding tube.

This inverted wick holder or washer-like valve 9 will be tightly bounded to the top portion of the lamp container cap to help prevent leakage or reflux of melted wax in the plate.

This inverted or washer—like device can be made from different materials, which include metals, glass, ceramics, the preferred material being aluminum.

The diameter of this wick holder-tube or washer system will be around 0.5-4 centimeters in diameter. The length can be anywhere from 0.1-4 centimeter. The preferred length will be 1.5 centimeter.

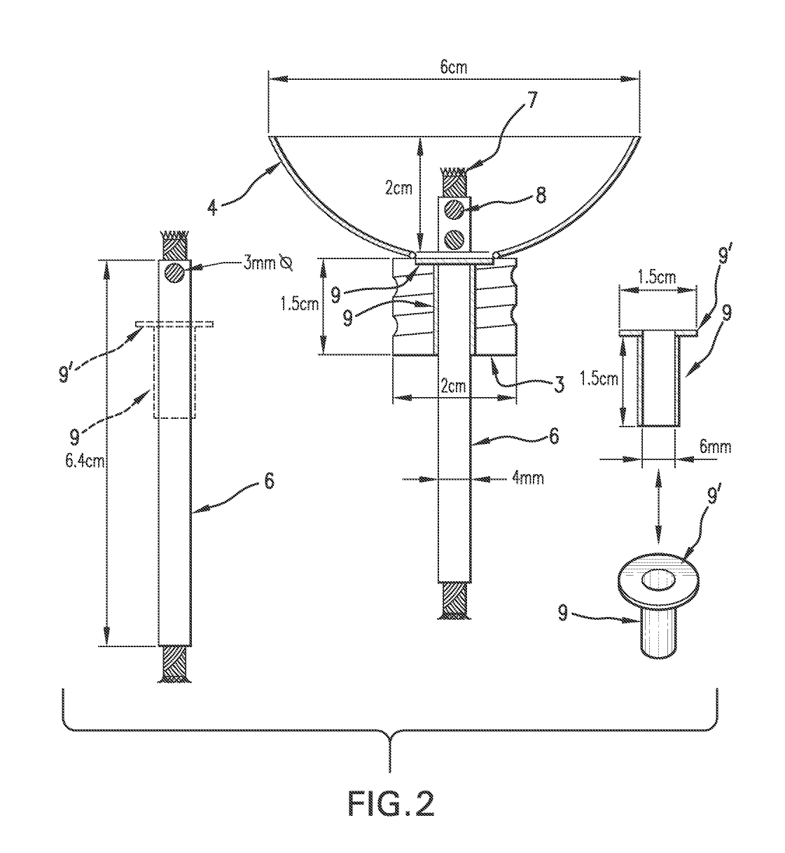

The various parts above-described are shown in more detail in FIG. 2, wherein a specific embodiment and its non-limiting dimensions are also detailed.

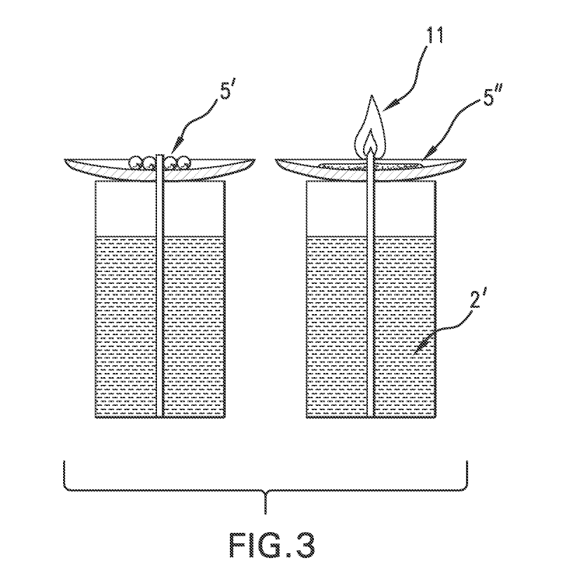

FIG. 3 represents another embodiment of the oil lamp according to the invention, wherein the device is shown prior to the combustion (left side of the picture) with the melting plate carrying a bead like wax material 5′ comprising the fragrance which, upon lighting of the wick supplied in fuel oil 2′ to produce the flame 11, melts to provide the fragrance diffusing melted material 5″ (right of the picture).

What is claimed is:

1. A device for the diffusion of light and of an active volatile substance, comprising a reservoir adapted to carry a liquid or solid fuel intended as a combustion oil supply for a combustible wick, said reservoir being provided with a capping piece having a hole or aperture adapted to receive a lodging element for the combustible wick, said wick being partially contained in the solid or liquid fuel and having an upper end intended for providing the flame and extending from the cap's hole and, wherein:
i) the device further comprises a melting plate for carrying a meltable material containing said active volatile substance, said plate being lodged on top of the reservoir's capping piece;
ii) the device comprises a reflux protection element lodged across the hole or aperture of the capping piece and shaped so as to entirely cover the capping piece's hole and prevent liquid reflux into the reservoir; and
iii) a conductive element is provided in the near vicinity of the upper end of the wick which provides the flame and maintained in direct contact with the melting plate so as to provide conduction heat thereto.

2. The device according to claim 1, wherein the combustible fuel is devoid of said volatile substance carried in the meltable plate.

3. The device according to claim 1, wherein the meltable substance is a solid or gel candle material.

4. The device according to claim 1, wherein the reflux protection element is formed by the upper part of the lodging element for the combustible wick.

5. The device according to claim 4, wherein the reflux protection element is formed of a glass, ceramic or aluminum material.

6. The device according to claim 1, wherein the meltable material is a wax formed of paraffin, soy wax, soy/paraffin mixture, montan wax, carnauba wax, beeswax, microcrystalline wax or fatty acid and fatty acid ester based.

7. The device according to claim 1, wherein the meltable material is a wax formed of paraffin, soy wax, soy/paraffin mixture, montan wax, carnauba wax, beeswax, microcrystalline wax or fatty acid and fatty acid ester based, or a polyamide gel.

8. The device according to claim 1, wherein the melting plate is formed of aluminum.
